(19) United States
(12) Patent Application Publication
(10) Pub. No.: US 2003/0004372 A1
(43) Pub. Date: Jan. 2, 2003

(54) Process for the recovery of crude terephthalic acid (cta)

(76) Inventors: Luciano Piras, Milan (IT); Sergio Schena, Mantova (IT); Michelle Chiarelli, Landriano (IT); Luigi Soro, Ghilarza (IT)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/181,534

(22) PCT Filed: Nov. 30, 2000
(86) PCT No.: PCT/IB00/01888

(51) Int. Cl. C07C 51/42

(52) U.S. Cl. 562/486

(57) ABSTRACT

The present invention relates to a process for the preparation of crude terephthalic acid which comprises first introducing a slurry containing crystallized terephthalic acid with impurities in an aliphatic carboxylic acid mother liquor into a rotary filter comprising a case and a rotating drum, wherein the rotating drum is subjected to a uniform pressure differential over the entire drum, and the inside of the drum is at a pressure approximately equal to atmospheric pressure or greater, then filtering said slurry; and finally collecting at least some of the solid portion. Other aspects of the invention include continuously adding the original slurry to the rotary filter and washing the solid portion with additional aliphatic carboxylic acid, water or a combination of the two. The solid portion can then be transferred to a reslurry tank which is at the same pressure as the pressure at the point where the solid portion was collected. The present invention also relates to a process for the preparation of crude terephthalic acid which comprises first introducing a slurry containing crystallized terephthalic acid with impurities in an aliphatic carboxylic acid mother liquor into a rotary filter comprising a case and a rotating drum, wherein the rotating drum is subjected to a uniform pressure differential over the entire drum, then filtering said slurry, washing the solid portion with water; and finally collecting at least some of the solid portion. Other aspects of the invention include continuously adding the original slurry to the rotary filter and additional washing with the aliphatic carboxylic acid mother liquor. The inside of the drum can be less than, greater than or approximately equal to atmospheric pressure. The solid portion can then be transferred to a reslurry tank which is at the same pressure as the pressure at the point where the solid portion was collected.

The present invention relates to a new process for the production and recovery of crude terephthalic acid (“CTA”). More particularly, this application relates to the use of a rotary pressure filter and water washing to recover crystalline terephthalic acid. The rotary filter is under a uniform pressure thus simplifying construction, maintenance and operation of the filter.

Crude Terephthalic Acid (CTA) is an intermediate product in the production of purified terephthalic acid, which is useful, for example, in the production of polyethylene terephthalate (PET). The CTA is produced by direct oxidation of p-xylene in a liquid solvent. The typical process for CTA recovery involves, among others, the steps of:

1. Crystallizing the CTA from the mother liquor of the oxidation reaction;

2. separating the crystalline CTA from its mother liquor (including the liquid reaction solvent, catalyst and unwanted by-products);

3. washing the solid so recovered with fresh solvent, to eliminate residual mother liquor contained in the CTA solid material;

4. drying the washed solid in a suitable dryer to remove the solvent and moisture;

5. storing the dry solid in storage silos for further processing.

The CTA is thereafter typically further processed to form a purified terephthalic acid (PTA) prior to its being used in other reactions, such as in the formation of PET.

The traditional method to separate the CTA from its mother liquor consists in filtering the slurry in a rotary vacuum filter (RVF) (at a temperature of from 80° C. to 100° C. and a pressure of from 0.3 to 0.7 bar). It is known in the art that an RVF consists of a rotary drum rotating inside a case. Vacuum is applied into the center of the drum (typically 0.3 to 0.7 bar) while the case is slightly under pressure (typically 1.05 to 1.4 bar). The resulting DP case/drum is accordingly typically in the range of from 0.35 to 1.1 bar. The drum is ideally divided into three zones:

a) mother liquor separation zone;

b) cake washing zone: and

c) solid discharge zone.

The crystals of CTA coming from the first zone contain only a small amount of by products and catalyst (the majority of the by-products and catalyst remain in solution), but do contain residual mother liquor (typically 10 to 15 percent). To get rid of these impurities, the crystals, in the second zone, are usually washed with additional solvent, typically in a ratio of 0.3 to 0.5 m³ solvent/MT CTA, to displace the mother liquor still entrained. The solid is then discharged from the filter in the third zone and typically sent to a screw feeding the dryer. The CTA, then containing a residual 10÷15 percent of solvent, is then dried, typically in a rotary dryer, and stored. These traditional methods therefore have high capital costs in that they require a vacuum pumps, large scale dryer as well as storage silos.

The mother liquor, containing most of the by products and catalyst as well as water (typically in a range of 8 to 12 percent by weight) is partly recycled back to the oxidation reactor and partly sent to a solvent recovery section. Water content in the mother liquor recycled back to the oxidation reactor represents a critical item as it can negatively affect the oxidation reaction. Water concentration is also a critical item in the mother liquor which is sent to a solvent recovery system. The solvent recover system involves first removing the by-products and then distilling the remaining solvent. Those skilled in the art will recognize that the operating cost connected to this operation is directly proportional to the water content.

U.S. Pat. No. 5,200,557 (by Amoco) teaches a method of displacing with water, in counter-current stages, an aliphatic carboxylic acid (such as acetic acid) from a filter cake of an aromatic carboxylic acid (such as terephthalic acid) comprising pressure filtering. This reference teaches introducing a slurry of aromatic carboxylic acid (such as terephthalic acid) in a mother liquor comprising an aliphatic carboxylic acid (such as acetic acid) into the cake formation chamber of a rotating pressure filter comprising a number of filter cells. Following the rotation, the filter cells with the resulting filter cake are then transported into a wash chamber section where a stream of water typically heated to 85 to 95° C. is introduced into the filter cells to form a reservoir of water over the filter cake. Displacement washing is then achieved by forcing the water through the cake at a pressure gradient, which is in the range of 0.03 to 4.15 bar above the system pressure, while maintaining the reservoir. After the washing stages the filter cells are then transported into a drying chamber where a compressed inert gas is continuously introduced, at the proper pressure, in order to remove excess water from the cake. The filter cake is then recovered in the discharge chamber, while the filter cells, after rinsing with additional water, enter the cake formation chamber and repeat the process. This process reportedly results in cake that typically contains 12-14 percent by weight of water and less than 5000 ppm by weight of acetic acid, suitable for the purification stage. The rotating pressure filter having different filter cells requires complicated machinery which increases cost and the amount of downtime associated with maintenance of the equipment.

International Patent WO94/17892 (by ICI) teaches a method of displacing with water in counter current stages, acetic acid from a filter cake of terephthalic, acid filtering the slurry coming from the crystallizers by means of a belt filter at a temperature typically comprised between 80 and 150° C. in a housing enclosing an inert gaseous atmosphere at a pressure typically comprised between 3 and 15 bar. The cake is formed in the first section of the belt by the removal of the main part of the solvent mother liquor and then washed in the following sections of the belt in counter-current, starting with the pure washing medium (typically water). A typical pressure drop of 0.6 bar is achieved by suction, in order to force the washing medium and the gas to pass through the filter cake. Also the gas flow is fed in counter-current, in order to achieve an enhanced removal of acetic acid. The gas may be recycled, after a suitable treatment to remove saturation acetic acid therefrom. A cake with a reduced content of acetic acid, suitable for the purification stage is achieved. The belt filter having different filtering zones requires complicated machinery which increases cost and the amount of downtime associated with maintenance of the equipment as requiring a larger lay-out to accommodate the length of the belt filter.

Accordingly, it would be desirable to have a new procedure for the collection and isolation of CTA which did not require expensive equipment like vacuum pumps, dryers and large silos, did not require expensive equipment like multi-zoned belt or rotational pressure filters, and minimized the amount of water contained in the mother liquor or spent solvent. The applicants of the present invention have found that the process can be improved by using a Rotary Pressure Filter (RPF) to separate the CTA crystals from the mother liquor. A rotary pressure filter differs from the approach in U.S. Pat. No. 5,200,557 in that the filtering medium is subjected to a uniform pressure differential. The case of the RPF is at the pressure slightly higher than that of the last crystallizer (preferably from 1.1 to 7.2 bar) and preferably operates at a temperature of from 110° C. to 180° C. It was also discovered that no special devices to discharge the cake to atmospheric pressure is required. It was also discovered that the CTA could be washed with water so that the resulting solids could then be transferred directly to the purification stage without being dried or stored. It was also discovered that the use of the RPF allows the amount of water used in the wash to be minimized so that the water concentration in the solvent to be recovered is also minimized.

One aspect of the invention is a process for the preparation of crude terephthalic acid which comprises first introducing a liquid slurry containing crystallized terephthalic acid with impurities into a high pressure rotary filter then filtering the slurry with a high-pressure rotary filter, wherein the filter is subjected to a uniform pressure differential, and then collecting at least some of the solid portion. Then the solid portion is washed with additional amounts of solvent and/or water. The washed solid portion is then transferred to a discharge zone, in which the solid portion is detached from the filter media. The solid collected from the high pressure rotary filter can then be discharged into a reslurry tank in which water is added to form a slurry having a strength suitable to be pumped to the tank feeding a purification section.

The slurry of terephthalic acid containing the impurities can come from any terephthalic acid production scheme. These schemes are known in the art and are of minimal importance to the invention at hand. The particular high-pressure rotary filter used to filter the slurry is similarly not critical to the present invention. Any filtering system capable of operating at a pressure greater than atmospheric pressure may be used. Preferably, for standard operating conditions, the filtration area is large enough to allow handling of the full throughput of terephthalic acid of the plant, and capable of operating under pressures of from one to eight bar. A suitable filter is the Bird Young Rotary Filter sold by Baker Process Inc. A particular filter suitable for use in the apparatus of the present invention is a Bird Young Rotary Filter having a filtration area of 1 sq. ft, which is able to process from 1 to 3 MT/h of solid CTA.

Rotary Pressure Filters basically consist of a case, which is preferably pressurized at process pressure, and a drum covered by a filtering device such as a cloth or equivalent filtering device, which is pressurized at a pressure suitably lower than the case.

The drum rotates, and as it rotates each point ideally passes through three zones:

a first zone, where the mother liquor is removed;

a second zone, where the solid is washed with solvent and/or water and the liquid wash is removed; and

a third zone, where the solid is discharged.

To simplify the operation, the pressure of the case is preferably slightly higher than the pressure of the process step immediately preceding the pressure filter, and is thus typically in the range of 1.3 to 7.4 bar, preferably in a range of from 3.0 to 6.5 bar, with about 4.5 bar being most preferred. The filtering is carried out at a temperature in the range of from 110 to 180° C., preferably in a range of from 144 to 175° C., with about 160° C. being most preferred.

The pressure difference between case and the center of the drum should be in the range of from 0.05 to 1.1 bar, preferably in a range of from 0.3 to 0.7 bar, with about 0.5 bar being most preferred.

The mother liquor removed from the rotary pressure filter can be recovered and directly reused into the production process or sent to a solvent recovery section to separate the solvent from the by products, as is known in the art.

The remaining solid portion rotates on the drum to a zone where it is optionally washed with additional amounts of solvent. While any amount of solvent may be used in the washing stage of the present invention, it is preferred that less than 1.0 m3/MT of CTA be used, in order to reduce the energy associated to heating up this solvent to process temperature, and the costs involved with treating the spent solvent. The solvent amount is preferably in a range of from 0.1 to 0.5 m3/MT of CTA, more preferably in a range of from 0.2 to 0.4 m3/MT of CTA, with about 0.3 m3/MT of CTA being most preferred. The washing is preferably done at the same temperature as the filtering, although this is for convenience and is not mandatory. The temperature of the solvent used to wash the solid material should be in the range of from 50 to 180° C., preferably in the range of 80 to 150° C., most preferably about 110° C. Preferably, the wash is accomplished in a counter current manner such that there are two or more wash zones and the purest wash liquid is used in the wash zone nearest the point at which the solid portion is collected, and the filtrate from each wash zone is recycled to be used in a wash zone closer to the point at which the slurry is introduced to the drum. This counter current washing is generally known in the art. The wash liquor can then be combined with the mother liquor or be separately collected and reused into the production process or recycled back into the production process, as is known in the art.

The remaining solid portion is then washed with additional amounts of water. While any amount of water may be used in the washing stage of the present invention, it is preferred that less than 0.5 m3/MT of CTA be used, in order to reduce the water content in the spent wash. The water amount is preferably in a range of from 0.05 to 0.2 m3/MT of CTA, with about 0.15 m3/MT of CTA being most preferred. This last washing is preferably done at the same temperature as the filtering, although this is for convenience and is not mandatory. The temperature of the water used to displace the solvent from the solid material should be in the range of from 50 to 166° C., preferably in the range of 100 to 160° C., most preferably about 147° C. The spent wash can then be combined with the mother liquor and/or solvent wash or separately collected, as is known in the art.

In the alternative where the washing is performed with water only, it is preferred that slightly more water be used, but it is still preferred that the amount be less than 1.0 m3/MT of CTA, more preferred that it be in a range from 0.2 to 0.4 m3/MT of CTA, with about 0.3 m3/MT of CTA being most preferred

After water washing, the solid material is collected into a discharge zone where it is can be further processed. Preferably, the solid CTA is passed by gravity into a reslurry tank, which is at the same pressure as the discharge zone. After the solid has moved into the reslurry tank, water is added to bring the slurry strength to a value in the range of from 5 to 60 percent on a weight basis, preferably in the range of from 20 to 50 percent on a weight basis, most preferably in the range of from 26 to 31 percent on a weight basis. The suitable temperature of the added water shall be calculated to obtain a slurry temperature in the reslurry tank lower than 166° C., preferably in the range of from 70 to 147° C., most preferably in the range of from 90 to 130° C. The slurry from the reslurry tank is then pumped out to the purification section where the hydrogenation of the crude terephthalic acid to produce PTA, as known in the art, is performed.

What is claimed is:

1. A process for the preparation of crude terephthalic acid which comprises:
a. introducing a slurry containing crystallized terephthalic acid with impurities in an aliphatic carboxylic acid mother liquor into a rotary filter comprising a case and a rotating drum, wherein the rotating drum is subjected to a uniform pressure differential over the entire drum;
b. filtering said slurry;
c. washing the solid portion with water; and
d. collecting at least some of the solid portion.

2. The process of claim 1 further comprising washing the solid portion with additional amounts of the aliphatic carboxylic acid mother liquor prior to washing with water.

3. The process of claim 1 or 2 wherein the wash is accomplished in a counter current manner such that there are two or more wash zones and the purest wash liquid is used in the wash zone nearest the point at which the solid portion is collected, and the filtrate from each wash zone is recycled to be used in a wash zone closer to the point at which the slurry is introduced to the drum.

4. The process of any one of claims 1 to 3 wherein the inside of the drum is at a pressure which is greater than the atmospheric pressure.

5. The process of any one of claims 1 to 3 wherein the inside of the drum is at a pressure approximately equal to atmospheric pressure.

6. The process of any one of claims 1 to 3 wherein the inside of the drum is at a pressure less than atmospheric pressure.

7. A process for the preparation of crude terephthalic acid which comprises:
a. introducing a slurry containing crystallized terephthalic acid with impurities in an aliphatic carboxylic acid mother liquor into a rotary filter comprising a case and a rotating drum, wherein the rotating drum is subjected to a uniform pressure differential over the entire drum, and the inside of the drum is at a pressure approximately equal to atmospheric pressure or greater;
b. filtering said slurry; and
c. collecting at least some of the solid portion.

8. The process of claim 7 wherein the solid portion is washed after being filtered in step b.

9. The process of claim 8 wherein the wash is with additional amounts of the aliphatic carboxylic acid mother liquor.

10. The process of claim 8 wherein the wash is water.

11. The process of claim 8 wherein the solid portion is washed first with additional amounts of the aliphatic carboxylic acid mother liquor and then with water.

12. The process of any one of claims 1 to 11 wherein the slurry containing crystallized terephthalic acid with impurities in an aliphatic carboxylic acid mother liquor is continuously introduced to the rotary filter.

13. The process of any one of claims 1 to 12 further comprising:
passing the collected solid portion to a reslurry tank wherein the reslurry tank is at a pressure not higher than the pressure where the solid portion was collected; and
mixing the solid with water to form a slurry of desired consistency for further processing.

14. The process of claim 13 wherein the solid concentration in the reslurry tank is in the range of from 5 to 60 percent on a weight basis.

15. The process of claim 14 wherein the solid concentration in the reslurry tank is in the range of 20 to 50 percent on a weight basis.

16. The process of claim 15 wherein the solid concentration in the separate tank is, in the range of 26 to 31 percent on a weight basis.

17. The process of any one of claims 2 to 6, 9 or 11 to 16, wherein the temperature of the wash solvent is in the range of from 50 to 180° C.

18. The process of claim 17 wherein the temperature of the wash solvent is in the range of from 80 to 150° C.

19. The process of claim 18 wherein the temperature of the wash solvent is 110° C.

20. The process of any one of claims 2 to 6, 9, or 11 to 19, wherein the solid portion is washed at a rate of less than 1.0 m³ of solvent per ton of solid.

21. The process of claim 20 wherein the solid portion is washed at a rate in the range of from 0.2 to 0.4 m³ of solvent per ton of solid.

22. The process of claim 21 wherein the solid portion is washed at a rate of 0.3 m³ of solvent per ton of solid.

23. The process of any one of claims 1 to 6 or 10 to 16, wherein the temperature of the added water is in the range of from 50 to 166° C.

24. The process of claim 23 wherein the temperature of the added water is in the range of from 100 to 160° C.

25. The process of claim 24 wherein the temperature of the added water is 147° C.

26. The process of any one of claims 1 to 6, 10 to 16 or 22 to 25, wherein the solid portion is washed at a rate in the range of less than 0.5 m³ of added water per ton of solid.

27. The process of claim 26 wherein the solid portion is washed at a rate in the range of from 0.05 to 0.2 m³ of added water per ton of solid.

28. The process of claim 27 wherein the solid portion is washed at a rate of 0.15 m³ of added water per ton of solid.

29. The process of any one of claims 1 to 28 wherein the temperature is released in a separate tank.

30. A process for the preparation of crude terephthalic acid which comprises:
a. introducing a liquid slurry containing crystallized terephthalic acid with impurities into a high pressure rotary filter;
b. filtering said slurry and collecting at least some of the solid portion;
c. washing the solid portion with water;
d. collecting the washed solid portion from the discharge zone;
e. passing the collected washed solid portion to a reslurry tank, wherein the reslurry tank is at a pressure not higher than the zone in which the solid portion was collected;
f. diluting the collected washed solid portion with additional amounts of water to form a slurry of a suitable strength;
g. pumping said slurry to the purification section;
h. collecting the mother liquor and the wash water.

31. The process of claim 30 wherein the solid concentration in the reslurry tank is in the range of 5 to 60 percent on a weight basis.

32. The process of claim 31 wherein the solid concentration in the reslurry tank is in the range of 20 to 50 percent on a weight basis.

33. The process of claim 32 wherein the solid concentration in the separate tank is, in the range of 26 to 31 percent on a weight basis.

34. The process of claims 30 to 33 wherein the temperature of the wash water is in the range of from 50 to 166° C.

35. The process of claim 34 wherein the temperature of the wash water is in the range of from 100 to 160° C.

36. The process of claim 35 wherein the-temperature of the wash water is 147° C.

37. The process of any one of claims 30 to 36 wherein the solid portion is washed at a rate in the range of less than 1.0 m³ of wash water per ton of solid.

38. The process of claim 37 wherein the solid portion is washed at a rate in the range of from 0.2 to 0.4 m³ of wash water per ton of solid.

39. The process of claim 38 wherein the solid portion is washed at a rate of 0.3 m³ of wash water per ton of solid.

40. The process of any one of claims 30 to 39 wherein the temperature is released in a separate tank.
